(19) United States
(12) Patent Application Publication
(10) Pub. No.: US 2004/0081706 A1
(43) Pub. Date: Apr. 29, 2004

(54) Alginate containing antimicrobial composition

(76) Inventors: Eilidh Trainer, Ayrshire (GB); Thomas Gilchrist, Ayrshire (GB)

Correspondence Address: Daniel A. Monaco, Esq., DRINKER BIDDLE & REATH LLP, One Logan Square, 18th and Cherry Sts., Philadelphia, PA, 19103-6996, US

(21) Appl. No.: 10/425,411

(22) Filed: Apr. 29, 2003

Related U.S. Application Data
(63) Continuation of application No. 09/142,776, filed on Sep. 11, 1998, now abandoned.
Application No. 09/142,776 is a 371 of international application No. PCT/GB97/00715, filed on Mar. 13, 1997.

(30) Foreign Application Priority Data
Mar. 13, 1996 (GB) 9605247.7

(51) Int. Cl. A61K 33/38; A61K 33/06; A61K 31/729

(52) U.S. Cl. 424/618; 424/682; 514/54

(57) ABSTRACT

There is provided a composition comprising an admixture of a finely divided alginate (or a salt or derivative thereof) together with a finely divided carrier material. The composition overcomes the problems associated with applying gel-forming alginates to a body surface without formation of a clumpy paste that leads to local irritation. An admixture of sodium alginate and a water-soluble glass carrier material is preferred. Optionally, the alginate and carrier material each have a particle size of less than 150 μm diameter and are present in a weight ration of 20:30 to 80:20. The presence of the carrier aids even gel formation and also promotes wound healing.

The present invention relates to an anti-microbial composition for use in medical or veterinary applications.

A wide variety of gels, creams, ointments, lotions etc are available for application to a body surface. The exact content of such compositions generally depends upon the purpose of application which may be, for example, to clean a body surface, to promote healing of any wound or injury, to prevent an exposed area of the body from drying out, to prevent infection etc. In certain circumstances the composition may include an active ingredient which is administered to the patient by application of the composition.

One example of a commercially available gel is INTRASITE™ produced by Smith & Nephew Ltd. This hydrogel contains hydrated carboxymethylcellulose as its main ingredient, and is applied to wounds in gel form as a primary treatment in order to clean the exposed surface by aiding removal of cell debris, dirt etc. In addition to acting as a sloughing agent, the gel also keeps the wound from drying out, thereby promoting healing.

Another example of a gel suitable for use on a wound dressing is described in EP-A-0586260 of Courtaulds Fibres Ltd. The gel disclosed is an alginate gel having an alginate content of 2 to 11 percent by weight.

Surgical dressings based on gel forming alginates have a significant contribution to make in wound management and are generally presented as preformed components of gels and pastes and as fibres of calcium or mixed calcium/sodium salts.

In alginate-based surgical dressings tire starting raw material is usually the sodium salt which is supplied by the alginate producer as a dry powder. Attempts to utilise alginate as topical powders for direct application to wounds have not proved successful. This is because the irregularly dispersed powder does not wet easily and clumping occurs leading to clusters of dry particles which can be sites of local irritation. There is incomplete gelling as a result and the desired sealing of the wound with a smooth hydrogel coating is not achieved.

It has now been found that an admixture of finely divided alginate (the term “alginate” being used herein to refer to alginates, the derivatives and salts thereof) and a different finely divided carrier material can be applied to wounds or other moist body surfaces. The combination of the carrier material together with the alginate facilities the formation of an even gel coating and the avoidance of clumping.

Suitable carrier materials include proteins (eg casein), salts (eg sodium, zinc, calcium, magnesium and potassium salts) and water-soluble glass. Desirably the carrier material is water-soluble or water miscible.

More surprisingly, it has been found that the alginate/carrier combination acts in synergy to promote healing and cell growth. For example, in animal implant studies which compared alginate powder alone and a water-soluble glass powder alone with a blend of both, it was demonstrated that tissue response was clearly better for the mixed powders than that seen with either material on its own. In particular at 14 days after implantation there was little evidence of the inflammatory cells which were residually present in the single material implant sites.

Viewed from one aspect the present invention provides an admixture of alginate or a derivative or salt thereof together with a carrier material. Generally both main components are finely divided, i.e. are in powder, particulate or granular form.

Desirably the finely divided alginate and carrier material components may each have a diameter size of 150 μm or less. Preferably the mode particle size for either component is 100 μm or less. More preferably the mode particle size for either component is 60 μm or less, for example 30-60 μm.

The two components may be combined together in any suitable mixture. Suitable mixtures include those having a ratio of from 20:80 to 80:20 (% by weight) of alginate:carrier. Preferred mixtures include those having an alginate:carrier ratio in the range of 20:80 to 50:50, preferably 20:80 to 30:70, for example 25:75.

Water-soluble glasses are a preferred form of carrier material. The use of glasses which can dissolve in water and body fluid and which are applied internally of the body are well-known. These glasses are formed from phosphorus pentoxide and may be modified to dissolve over a period of minutes, months or even years, as required. To date, such glasses have been used, in medicine, for the controlled release of a number of agents, for example, drugs, hormones and trace elements, but in each case the glass has been applied internally of the body to allow the agent to leach out into the body's circulatory system.

It is known that certain glasses, in which the usual glass former, silicon dioxide, of traditional glasses is replaced with phosphorus pentoxide as the glass former, are soluble in water and body fluids. The rate of dissolution is controlled largely by the addition of glass modifiers such as calcium and magnesium oxide. In simple terms, the greater the concentration of the modifier the slower is the rate of dissolution. The rates of dissolution which can be imparted to the glasses may range from minutes to months or even to several years. It is known to include in such compositions quantities of trace elements such as copper, cobalt and selenium which will be released from the glass as it slowly dissolves over the selected period of time.

The use of water-soluble glasses has been described for a variety of purposes in the literature. For example, UK Patent Specifications numbers 1,565,906, 2,079,152, 2,077,585 and 2,146,531 describe the gradual dissolution of the glasses as providing a means of controlled release of drugs, hormones, fungicides, insecticides, spermicides and other agents with which the glasses have been impregnated. The glasses are used for example in the form of an implant or bolus.

UK Patent Specification number 2,030,559 describes the use of selenium-impregnated water-soluble glass for providing controlled release of the selenium as a trace element into cattle and sheep, the glass being applied as a subcutaneous insert. UK Patent Specification number 2,037,735 also describes a subcutaneous implant of water-soluble glass, and in this case the glass is impregnated with copper; minor quantities of trace elements such as boron, arsenic, iodine, manganese, chromium, silver, gold and gallium may also be included.

Water-soluble glass has also been proposed for use in prosthetics, for example in UK Patent Specification number 2,099,702, and for use in anticorrosive paints, as described in UK Patent Specification number 2,062,612. Further the literature provides for the use of such glasses in the controlled release of ferrous and ferric ions into the human or animal body by ingestion or implantation of the glass (UK Patent Specification number 2,081,703), and for the use of glasses in the controlled release of ions such as lithium, sodium, potassium, caesium, rubidium, polyphosphate, calcium and aluminium to patients by inclusion of the glass in a drip feed line (UK Patent Specification number 2,057,420).

WO-A-89/01793 relates to apparatus for antimicrobial use in passage of fluid to or from a living body, the apparatus comprising a conduit for insertion into the body, a reservoir for fluid and a connector member for connecting said conduit to said reservoir external of the body, wherein said connector member includes a water-soluble glass impregnated with elemental silver or a compound of silver, said water-soluble glass defining at least a part of a passageway for fluid to flow between the reservoir and the conduit.

Desirably the water-soluble glass is a silver containing water-soluble glass. Advantageously the silver content will be introduced into the glass composition in the form of silver orthophosphate.

Suitable glasses include, for example, the ARGLAES™ glass of Giltech Limited.

Preferably, said glass is adapted by the use of glass modifiers to give a sustained release of silver ions over a set period.

In one embodiment the water-soluble glass comprises an alkali metal oxide M₂O, an alkaline earth oxide MO, phosphorus pentoxide P₂O₅ and silver oxide (Ag₂O) or silver orthophosphate (Ag₃PO₄).

Most preferably, said glass contains not more than 40 mole % M₂O or MO, not less than 10 mole % M₂O or MO, and not more than 50 mole % nor less than 38 mole % phosphorus pentoxide, with the inclusion of 0.05 to 5.0 mole % silver oxide or orthophosphate.

Said alkali metal oxide may be sodium oxide (Na₂O), potassium (K₂O) or a mixture thereof; and said alkaline earth oxide may be calcium oxide (CaO), magnesium oxide (MgO), zinc oxide (ZnO) or a mixture thereof.

The glass may also contain less than 5 mole % silicon dioxide (SiO₂), boric oxide (B₂O₃), sulphate ion (SO₄²⁻), a halide ion, copper oxide (CuO) or a mixture thereof.

Typically the soluble glasses used in this invention comprise phosphorus pentoxide (P₂O₅) as the principal glass-former, together with any one or more glass-modifying non-toxic materials such as sodium oxide (Na₂O), potassium oxide (K₂O), magnesium oxide (MgO), zinc oxide (ZnO) and calcium oxide (CaO). The rate at which the silver-release glass dissolves in fluids is determined by the glass composition generally by the ratio of glass-modifier to glass-former and by the relative proportions of the glass-modifiers in the glass. By suitable adjustment of the glass composition, the dissolution rates in water at 38° C. ranging from substantially zero to 25mg/cm²/hour or more can be designed. However, the most desirable dissolution rate R of the glass is between 0.01 and 2.0 mg/cm²/hour. The water-soluble glass is preferably a phosphate glass, and the silver may advantageously be introduced during manufacture as silver orthophosphate (Ag₃PO₄). The content of silver and other constituents in the glass can vary in accordance with conditions of use and desired rates of release, the content of silver generally being up to 5 mole %. While we are following convention in describing the composition of the glass in terms of the mole % of oxides, of halides and of sulphate ions, this is not intended to imply that such chemical species are present in the glass nor that they are used for the batch for the preparation of the glass.

The optimum rate of release of silver ions into an aqueous environment may be selected by circumstances and particularly by the specific function of the released silver. The invention provides a means of delivering silver ions to an aqueous medium at a rate which will maintain a concentration of silver ions in said aqueous medium of not less than 0.01 parts per million and not greater than 10 parts per million. In some cases, the required rate of release may be such that all of the silver added to the system is released in a short period of hours or days and in other applications it may be that the total silver be released slowly at a substantially uniform rate over a period extending to months or even years. In particular cases there may be additional requirements, for example it may be desirable that no residue remains after the source of the silver ions is exhausted or, in other cases, where the silver is made available it will be desirable that any materials, other than the silver itself, which are simultaneously released should be physiologically harmless. In yet other cases, it may be necessary to ensure that the pH of the resulting solution does not fall outside defined limits.

The glass may be formed by a number of methods. It may simply be cast by conventional or centrifugal procedures, or it may be prepared via one or more stages of rod, fibre or tube drawing. Other preparation techniques include foamed glass. Following glass formation it will be comminuted into finely divided form.

With regard to the alginate component, derivatives and salts of alginates are acceptable for use in the present invention. Sodium and calcium salts of alginate or a combination of these two salts is preferred. Sodium alginate is especially preferred.

In one preferred embodiment, the composition of the present invention is an admixture of sodium alginate powder and water soluble glass (eg ARGLAES™ of Giltech Limited) in a ratio of alginate:glass of 25:75 by weight. Preferably, the water soluble glass releases calcium ions as it dissolves. The calcium ions displace some of the sodium ions in the sodium alginate thus forming calcium alginate. The presence of calcium alginate stabilises the alginate gel.

The composition may be pre-mixed, or alternatively the alginate may be kept separately from the carrier material and the ingredients admixed together immediately prior to use. This enables a particular blend to be formulated to suit the wound or condition in question.

Optionally, the composition of the present invention may contain an active ingredient. The term “active ingredient” is used herein to refer to any agent which affects the metabolism or any metabolic or cellular process of the patient (including growth factors and living cells), promotes healing, combats infection, hypergranulation or inflammation. Antibiotics and other anti-bacterial agents, steroids, painkillers etc are all suitable. Optionally, the active ingredient may be in delay-release or controlled-release form.

The composition of the present invention may be used to clean a body surface, to promote healing of a wound or injury, to prevent an exposed area of the body from drying out or to prevent infection.

In a further aspect the present invention provides a method of treating the human or non-human (preferably mammalian) animal body, said method comprising applying a finely divided admixture of an alginate (a derivative or salt thereof) and a carrier material, such as a (preferably silver-containing) water-soluble glass, to a body surface, for example to a wound.

The invention will now be further described with reference to the figures:

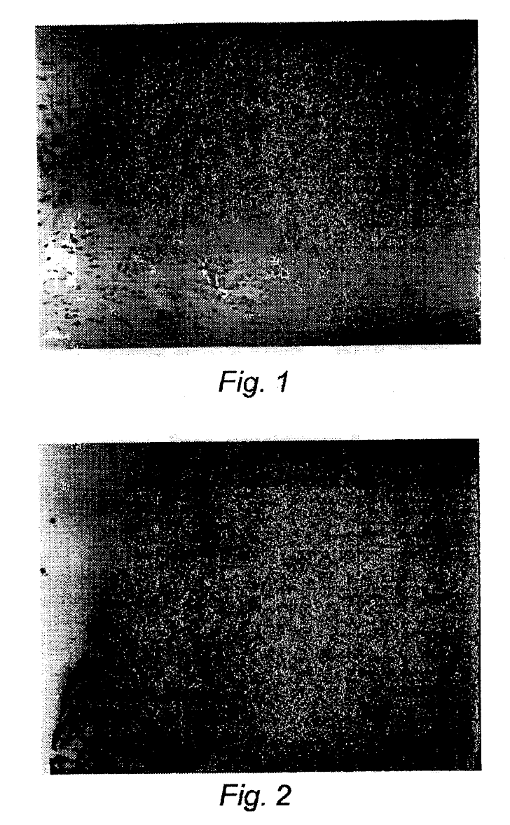

FIG. 1 illustrates a mass of inflammatory cells at the site of implantation of a composition of just silver ion releasing glass, 7 days after implantation.

FIG. 2 illustrates a mass of inflammatory cells and the damage to the muscle bed at the site of implantation of alginate, 2 days after implantation.

FIG. 3 is a higher magnification of the same tissue block as in FIG. 2.

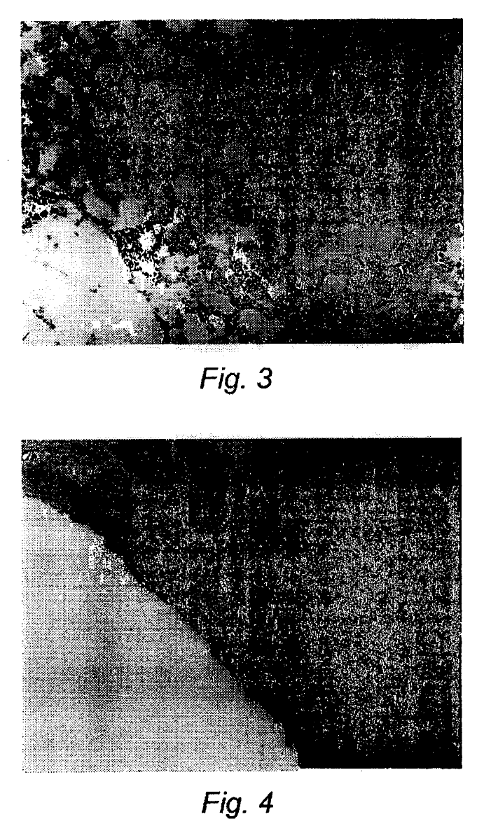

FIG. 4 illustrates a mass of inflammatory cells sitting on and infiltrating the muscle bed at the site of implantation of a composition of just alginate, 7 days after implantation.

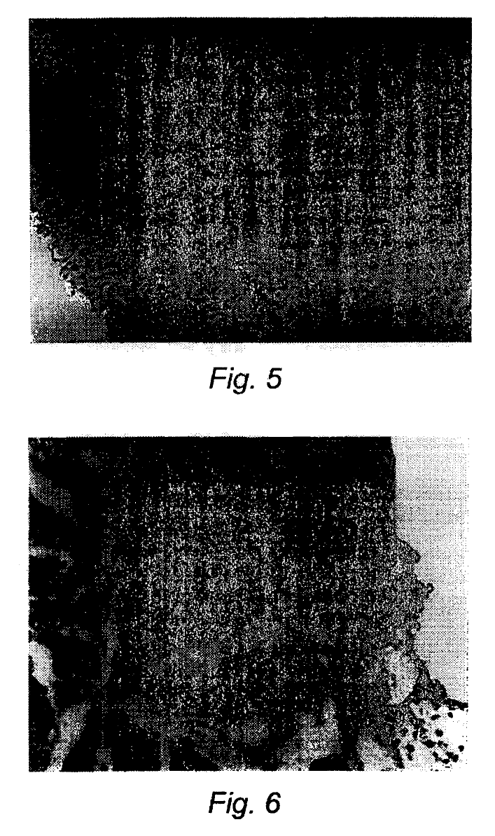

FIG. 5 is a higher magnification of the same tissue block as in FIG. 4.

FIG. 6 illustrates a number of inflammatory cells and the broken up muscle bed at the site of implantation of a composition of alginate and a water soluble glass carrier, 2 days after implantation.

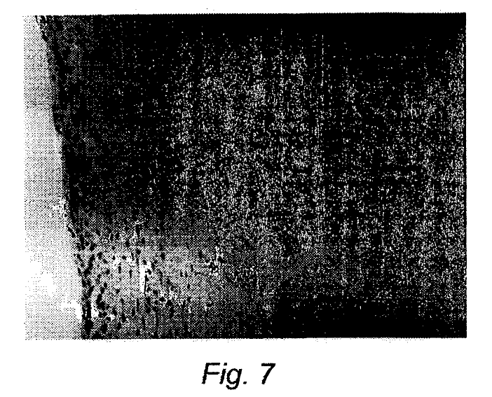

FIG. 7 illustrates a number of inflammatory cells and a normal muscle bed at the site of implantation of a composition of alginate and a water soluble glass carrier, 7 days after implantation.

and with reference to the following, non-limiting, examples.

### EXAMPLE 1

To determine the tissue response to the powdered biomaterials using a rat model and further to determine whether combining the two materials had a significant effect on the response.

| Materials | |
|---|---|
| CRG/silver powder [D301893 Ag 3 mole %] | CRG/Ag |
| Alginate powder [lot No 544831] | Alginate |
| CRG/silver powder and | Alginate/CRG/Ag |
| Alginate powder [50:50] mix | |

The silver containing controlled release glass (herein referred to as “CRG/silver”) had the following composition

| | | |
|---|---|---|
| Na₂O | 27.5 | mole % |
| CaO | 22 | mole % |
| Ag₂O | 3.5 | mole % |
| P₂O₅ | 47 | mole % |

The silver content of the glass was added in the form of silver orthophosphate, but is expressed as “silver oxide” according to convention. 100% of the glass particles had a diameter of less than 53 μm.

The alginate used was a pure sodium alginate salt, commercially available as Manucol™ LKX of Kelco International Limited, United Kingdom. The volume mode particle size of the sodium alginate is 41.46 μm and 99.4% of the particles had a diameter of less than 49.99 μm.

All materials were supplied in powder form. The Alginate/Ag mix was prepared by hand. The materials were not sterilised before implantation. No infection problems were encountered during the procedures.

Method

Adult, black and white hooded rats of the Lister strain (approximately 200 g) were used for all procedures. Appropriate surgical methods were employed by experienced personnel, and all procedures were carried out as detailed in UK Home Office licence No PP140/01099.

A small incision was made above the lumbar sacral vertebrae, and the muscle bed on either side of this incision was exposed by blunt dissection. A pocket was created in the muscle fibres and approximately 2 mg of the powdered material was carefully placed into this pocket. Inevitably, some powder material was deposited on the muscle bed surface and contacted subcutaneous tissue. Animals were sacrificed at 2, 7 and 14 days using a schedule one method.

Following sacrifice, the tissue was examined for any obvious signs of inflammation, and a block of tissue/muscle containing the implant site was removed. The block was immediately frozen, sectioned on a cryostat microtome to produce sections 7 μm thin and stained using haematoxylin and eosin. The sections were examined by light microscopy.

Results

CRG/Ag

2 days

There were no signs of gross inflammation when the animals were sacrificed. Following staining the site could be seen to be heavily inflamed. The muscle was widely infiltrated with neutrophils, and the muscle fibres were disrupted. A black particulate material (believed to be an Ag/Ag complex) was evident and neutrophils were very densely packed around these particles.

7 days

Although the muscle site appeared clean, there was a large volume of clear exudate present at each implant site. The exudate had produced a swelling under the skin at the site of the implantation. Following staining, a mass of inflammatory cells were seen to be present at the site (FIG. 1). These cells appeared to be predominantly neutrophils. The muscle fibres appeared normal and there was no evidence of necrotic tissue, though there remained some inflammatory infiltration. Particulate matter was present though not black in this case. It looked more like a degrading glass. The silver could not be detected at this time.

14 days

The exudate and associated swelling had subsided by this time, however when the site was exposed there was evidence of tissue damage (believed to be necrosis) on the muscle bed and in contiguous subcutaneous tissue. Following staining extensive inflammation was apparent, and there was evidence of necrotic tissue. However, only a small area was affected. Some dark, particulate material was also evident. This may be a silver complex. Degrading glass material is clearly present at the site.

Alginate

2 days

No gross signs of inflammation were present when the animals were sacrificed. However, the alginate was clearly visible on and around the implant site as a “messy” gel. Following staining, large numbers of inflammatory cells could be seen (FIG. 2), the muscle bed was damaged and the muscle fibres were disturbed and infiltrated with these cells. This was possibly due to the presence of small particulate material invading the muscle and stimulating an inflammatory response. However, there was no evidence of necrotic response.

FIG. 3 shows a higher magnification of the response from the same tissue block as FIG. 2. Inflammatory cells can be seen invading the muscle fibres. Most of the pink stained material visible was alginate, clearly well dispersed. Muscle fibres (also stained pink) could be seen in the top right corner. Alginate could be seen, stained pink.

7 days

No signs of gross inflammation were evident when the animals were sacrificed. No alginate could be seen at this time, and the muscle bed appeared clean. Following staining (FIG. 4), large numbers of inflammatory cells could be seen remaining at the implant site. However, there was very little evidence of alginate remaining at the site even when the site was observed under higher magnification (FIG. 5). The result was very similar to that observed with the Ag at 7 days although in this case there was no exudate build-up.

14 days

No sign of gross inflammation was present when the animal was sacrificed. Following staining, large numbers of inflammatory cells were evident at the implant site. There was some evidence of alginate remaining at the site, but only very little. There was no evidence of necrosis or damage to the tissue.

Alginate/CRG/Ag

2 days

There were no gross signs of inflammation when the animals were sacrificed, and the muscle bed appeared clean. Following staining (FIG. 6), the muscle fibres could be seen to be disturbed and the muscle bed to be broken up. This was likely to be due to the particulate matter stimulating infiltration of inflammatory cells. However, there appeared to be fewer inflammatory cells at the implant site or infiltrating the muscle than was evident when the materials were examined alone. There was only little evidence of particulate material remaining at the site. Once again, this appeared to be a degrading glass.

7 days

There were no gross signs of inflammation when the animals were sacrificed. Following staining (FIG. 7), large numbers of inflammatory cells could be seen at the implant site. There was some particulate material present, though it was not clear what this was. The response was similar to that seen at 2 days. However, the muscle bed now seems normal with the muscle fibres intact. The result was very similar to that seen with the materials examined alone at the same time period.

14 days

There were no signs of gross inflammation at the implant site following sacrifice. Staining showed a clean muscle block with only little evidence of inflammatory cells. The response at 14 days with the mixed materials, was clearly better than that seen with either material when examined alone. No evidence of any particulate material could be found at this time.

Conclusion

The majority of inflammation that is seen with these samples can probably be attributed to:

a. the surgical procedure itself; we are examining the tissue response within the normal wound healing time;

b. the fact that the material has been applied in power/particulate form; this will inevitably lead to a more extensive inflammation.

Nevertheless, differences have been noted in the responses to the materials examined. Silver containing CRG gave rise to a considerable exudate which was at its most severe, certainly most obvious at 7 days. This exudate was clearly visible under the skin as a lump, and the area was obviously painful to the animal. On sacrifice the exudate was revealed as a clear, subcutaneous fluid. At 14 days the exudate had subsided, although there remained a “sore” on the skin.

When exposed, the implant site, particularly the muscle bed surface and the subcutaneous tissue in contact with the implant site, was damaged. Histology showed that there was some evidence of necrotic tissue, though this was minimal.

The alginate alone produced a “messy” gel on the muscle surface at 2 days, but subsequent time periods showed a clean muscle bed. Inflammation was associated with the implant site at all time periods. However, there was no evidence of damage or necrotic tissue. Although the alginate is clearly dissolving, traces of alginate could still be found at the site for 14 days.

The alginate/silver mix seemed to attract less cells to the site at 2 days. At 7 days the response was fairly similar to that seen with the samples examined alone and no exudate was formed. However, after 14 days the healing response seemed much accelerated with this sample. Clean, normal muscle tissue was observed, with little evidence of inflammatory infiltration.

### EXAMPLE 2

Materials examined:

CRG/Ag powder

Alginate powder

Alginate/CRG/Ag powder

All the samples were implanted as powders.

Adult, black and white hooded Lister rats (approximately 200 g) were used.

A small incision was made above the lumber sacral vertebrae. A pocket was created in the muscle fibres and approximately 5 mg of the material was placed into the pocket. The wound was sutured with silk.

Two samples of each material were placed in each animal and two animals used for each time period. Animals were sacrificed at two and seven days.

At sacrifice the tissue was examined for any obvious signs of inflammation and a block of muscle containing the implant site removed. The block was frozen, sectioned on a microtome at 7 microns and stained by haematoxylin and eosin.

CRG/Ag Powder

2 days

There were no gross signs of inflammation when the animal was sacrificed. Following staining, the site could be seen to be heavily inflamed. The muscle was widely infiltrated with neutrophils, and the muscle fibres disrupted. A black particulate material (Ag/Ag complex) was in evidence and neutrophils were very densely packed around these particles.

7 days

Although the muscle site looked clean, there was a large volume of clear exudate present with each animal The exudate had produced a swelling under the skin at the site of the implant. Following staining, a huge mass of inflammatory cells were present at the implant site. These cells appear to be predominantly neutrophils. The muscle fibres looked normal, though there remained a considerably inflammatory cell infiltration. There was some particulate matter present, though not black in this case. It looked more like a degrading glass.

Alginate powder

2 days

No gross signs of inflammation when the animal was sacrificed, though the alginate was clearly visible on and around the implant site, as a “messy” gel. Following staining, large numbers of inflammatory cells could be seen and the muscle fibres were disturbed and infiltrated with these cells. Alginate could be seen, stained pink.

7 days

No gross signs of inflammation when the animal was sacrificed. No sign of alginate at this time. Muscle bed looked very clean. Following staining, large numbers of inflammatory cells could be seen remaining at the implant site, however, there was very little evidence of alginate remaining at the site. The result was similar to that observed with CRG/Ag at 7 days, although in this case there was no exudate build up.

Alginate/CRG/Ag

2 days

No gross signs of inflammation when the animal was sacrificed. The muscle bed was clean. Following staining, the muscle fibres could be seen to be broken up, however, there were less numbers of inflammatory cells at the implant site or infiltrating the muscle.

There was only little evidence of particulate material remaining at the site. Again this looked like a degrading glass.

7 days

No gross inflammation when the animal was sacrificed. Following staining large numbers of inflammatory cells could be seen at the site of implantation. Again there was some particulate material present (degrading glass). The muscle fibres were intact and normal.

### EXAMPLE 3

Method

Other powders have also been combined with alginate to establish whether a) these combinations also formed a gel and b) if any such gel was tacky.

The powders tried were casein, sodium chloride, zinc oxide, sodium borate, magnesium sulphate, magnesium chloride, calcium tetraborate and potassium iodide.

Each powder was admixed individually with sodium alginate (Manucol™ LKX) in a ratio of 3:1. The admixture was then applied to a damp simulated wound, covered with a dressing and left for 48 hours.

Results

Admixtures with casein, sodium chloride, magnesium sulphite, magnesium chloride and potassium iodide formed sticky but “lump free” gels.

Admixtures with zinc oxide and calcium tetraborate did not appear to wet out at ail.

The admixture with sodium borate did wet out adequately, but formed a rubbery coating on the simulated wound which did not stick to the dressing.

1. A composition comprising an admixture of sodium alginate and water soluble glass.

2. An admixture as claimed in claim 1, wherein the ratio of alginate:water soluble glass is in the range 20:80 to 80:20 by weight.

3. An admixture as claimed in claim 2, wherein the ratio of alginate:water soluble glass is 25:75 by weight.

4. A composition as claimed in claim 1, wherein said water soluble glass releases silver ions during dissolution.

5. A composition as claimed in claim 1, wherein said water soluble glass releases calcium ions during dissolution.

6. A composition as claimed in claim 1, wherein said alginate is finely divided and has a particle diameter of 150 μm or less.

7. A composition as claimed in claim 1, wherein said water soluble glass is finely divided and has a particle diameter of 150 μm or less.

8. A composition as claimed in claim 1, wherein said alginate and said water soluble glass each have a mode particle size of 60 μm or less.

9. A composition as claimed in claim 1, said composition comprising 75 parts by weight of a finely divided calcium ion releasing water soluble glass and 25 parts by weight of finely divided sodium alginate, said glass and said alginate each having a mode particle size of 60 μm or less.

10. A composition as claimed in claim 1, said admixture comprises a ratio of alginate:water soluble glass releasing silver ions in the range 20:80 to 80:20 by weight.

11. A method of treatment of a human or non-human animal body, said method comprising applying to a surface of said body a composition as claimed in claim 1.
